(19) United States
(12) Patent Application Publication
(10) Pub. No.: US 2022/0304602 A1
(43) Pub. Date: Sep. 29, 2022

(54) SYSTEM AND METHOD FOR HUMAN STRESS MONITORING & MANAGEMENT

(71) Applicant: Hitachi, Ltd., Tokyo (JP)

(72) Inventors: Xunfei ZHOU, Farmington Hills, MI (US); Subrata Kumar KUNDU, Canton, MI (US)

(73) Assignee: Hitachi, Ltd.

(21) Appl. No.: 17/214,375

(22) Filed: Mar. 26, 2021

(51) Int. Cl. A61B 5/16; A61B 5/18; A61B 5/00; G16H 50/70

(52) U.S. Cl. CPC A61B 5/165; A61B 5/18; A61B 5/4561; A61B 5/7264; G16H 50/70

(57) ABSTRACT

Example implementations described herein address limitations in related art human stress monitoring systems, as they are not application-aware, designed for a general purpose only, and have an undermined accuracy. The example implementations described herein utilize human posture as both a dynamic feedback mechanism and decision-making criteria to increase artificial intelligence model accuracy and recommend stress relaxing activities.

### BACKGROUND

### Field

The present disclosure is generally directed to human stress monitoring systems, and more specifically, to posture monitoring and adjustment systems to manage human stress.

### Related Art

Related art human stress monitoring systems are designed for general purpose only and are not customized to any specific application; thus, its accuracy is undermined. Moreover, the stress management function in the related art is either non-existing or very basic and lacks the application-specific way to reduce high-stress levels once they are detected.

In a related art implementation, there is a system that can be used to measure the driver stress levels in a vehicular environment. In such related art systems, the pulse rate is measured from electrodes placed on a steering wheel, and the respiratory rate is measured by a piezoelectric sensor placed on both seat belts and seat cover. A very specific feature extraction and calculation algorithm can be utilized by using pulse rate, respiratory rate, and vehicle speed to infer driver stress level.

In another related art implementation, a general device to monitor stress levels monitors the heartbeat interval time variation and relative stroke volume variation of a subject's heart via a sensor unit. Further, the stress level is determined by using a particular function involving heartbeat interval, stroke volume variation, and the mean stroke volume.

In another related art implementation, there are algorithms that involve sensor usage to heart rate, blood pressure, blood oxygen level, and human acceleration to classify stress levels. Other similar related art implementations utilize heartbeat, skin impedance, skin temperature, muscle cramp, and respiratory rate to infer stress. Such related art implementations can also involve using the sympathovagal balance (SVB) value to infer stress level, where SVB is calculated based on a set of measurement data that may include determining a heartrate variability (HRV) characteristic. Similarly, related art stress systems can also monitor stress based on heartrate, the HRV, and the activity level or provocation of a person.

### SUMMARY

Example implementations described herein involve a novel human stress monitoring and decision-making system that uses subject body posture information as a dynamic feedback mechanism and decision-making criteria that helps to increase system accuracy, as well as for recommending of stress relieving activities. Such an approach makes the stress monitoring and management system more personalized while catering to different application scenarios.

Stress adversely affects work performance and physiological states, causing economic and socially-significant problems. Monitoring those states is challenging because of the complex nature of the human body and the difficulties in designing a technique to accommodate everyday usage. In the related art, there have been numerous methods developed to monitor the stress state of a person, utilizing one or more approaches, including computer vision, photoplethysmography (PPG), Electroencephalography (ECG), skin conductance, and blood pressure measurements. However, these techniques/commercialized applications either have low performance or have no effective way to mitigate stress-introduced risks.

To solve the problem of low accuracy due to limited scene understanding of the traditional stress monitoring system, the example implementations described herein consider the effect of human body posture on stress level changes. The body posture can help the system understand what activities were conducted while feeling stressed and differentiate scenarios. For instance, human subjects tend to have varied facial expressions and vital signs while sitting and jogging, while the traditional system treats the data inputs from these two scenarios equally. Nevertheless, with the introduction of human posture recognition, a more refined stress level classification strategy can be executed based on understanding the current scenario. In the present disclosure, example implementations described herein dynamically change the artificial intelligence (AI) model architecture and ensemble classification voting weight based on the recognized posture.

Another issue to be solved in the related art is to recommend appropriate stress relaxing activities to manage the high-stress level and the associated risks. Similarly, such a recommendation requires scene understanding to suggest an action that will take less effort to conduct and effective at the same time. The example implementations described herein involve a stress-time-posture-person database to analyze how stress changes over time under different body postures. Once high stress is detected, the proposed system will search for the database for either an existing relaxing activity or search for most related activities related to the current posture.

Aspects of the present disclosure can involve a method, which can include determining a posture of a user from data provided from one or more sensors; determining a stress level of the user from a machine learning model configured to determine the stress level of the user based on the data provided from the one or more sensors; and for the determined stress level being above a threshold, determining one or more new postures to reduce the stress level from the database of postures; and recommending the one or more postures to the user.

Aspects of the present disclosure can involve a computer program, storing instructions for execution which can include determining a posture of a user from data provided from one or more sensors; determining a stress level of the user from a machine learning model configured to determine the stress level of the user based on the data provided from the one or more sensors; and for the determined stress level being above a threshold, determining one or more new postures to reduce the stress level from the database of postures; and recommending the one or more postures to the user. The instructions of the computer program can be stored in a non-transitory computer readable medium to be executed by one or more processors.

Aspects of the present disclosure can involve a system, which can include means for determining a posture of a user from data provided from one or more sensors; means for determining a stress level of the user from a machine learning model configured to determine the stress level of the user based on the data provided from the one or more sensors; and for the determined stress level being above a threshold, means for determining one or more new postures to reduce the stress level from the database of postures; and means for recommending the one or more postures to the user.

Aspects of the present disclosure can involve a apparatus, involving a processor, configured to determine a posture of a user from data provided from one or more sensors; determine a stress level of the user from a machine learning model configured to determine the stress level of the user based on the data provided from the one or more sensors; and for the determined stress level being above a threshold, determine one or more new postures to reduce the stress level from the database of postures; and recommend the one or more postures to the user.

### BRIEF DESCRIPTION OF DRAWINGS

FIG. 1 illustrates a sensor fusion stress monitoring system as implemented in the related art.

FIG. 2 illustrates an example of a neural network.

FIG. 3 illustrates an example AI model for a sensor fusion stress monitoring system, in accordance with an example implementation.

FIG. 4 illustrates a majority weight voting process as an example of ensemble, in accordance with an example implementation.

FIG. 5 illustrates examples of human body postures.

FIG. 6 illustrates an example implementation of a system architecture.

FIG. 7 illustrates an example of utilizing posture as dynamic feedback to the stress classification AI model feature fusion process, in accordance with an example implementation.

FIGS. 8(a) to 8(d) illustrate example changes to the AI model architecture under dynamic feedback from postures, in accordance with an example implementation.

FIG. 9 illustrates an example flowchart to recommend a relaxing posture adjustment based on the posture-stress-time database, in accordance with an example implementation.

FIGS. 10(a) to 10(c) illustrate a flowchart and an example for a similar posture search process, in accordance with an example implementation.

FIG. 11 illustrates the system architecture of the cloud-enabled human stress monitoring and management system, in accordance with an example implementation.

FIG. 12 illustrates the application aware stress monitoring and management as applied to driving, in accordance with an example implementation.

FIG. 13 illustrates the application aware stress monitoring and management as applied to desk working, in accordance with an example implementation.

FIGS. 14(a) and 14(b) illustrate an example processing application to determine driver states, in accordance with an example implementation.

FIG. 15(a) and FIG. 15(b) illustrates an example of an automatically adjustable seat and flow diagram, in accordance with an example implementation.

FIG. 16 illustrates a system involving a plurality of edge systems with connected sensors and a management apparatus, in accordance with an example implementation.

FIG. 17 illustrates an example computing environment with an example computer device suitable for use in some example implementations.

### DETAILED DESCRIPTION

The following detailed description provides details of the figures and example implementations of the present application. Reference numerals and descriptions of redundant elements between figures are omitted for clarity. Terms used throughout the description are provided as examples and are not intended to be limiting. For example, the use of the term “automatic” may involve fully automatic or semi-automatic implementations involving user or administrator control over certain aspects of the implementation, depending on the desired implementation of one of ordinary skill in the art practicing implementations of the present application. Selection can be conducted by a user through a user interface or other input means, or can be implemented through a desired algorithm. Example implementations as described herein can be utilized either singularly or in combination and the functionality of the example implementations can be implemented through any means according to the desired implementations.

Described herein are three aspects to utilize posture data as input in relation to stress levels. In a first aspect, posture data is used as additional input via a feedback mechanism to a machine learning system to increase the accuracy of the stress level measurement. In another aspect, a posture-stress-time database is utilized to determine postures that cause stress and as well as postures that are determined to relieve stress for recommendations. Depending on the desired implementation, the example implementations can be deployed in edge systems where the stress monitoring can be conducted, wherein the recommendations can be determined on the cloud system. Additionally, stress monitoring can also be conducted on the cloud, wherein the edge system is configured to collect the raw data only. The edge system can also be configured to conduct stress monitoring functions as needed. The management and monitoring functions described herein can be interchangeable in the edge system or the cloud system in accordance with the desired implementation.

Stress is one of the major attributes of mental health, and it has received growing interest from both industry and academia. Numerous studies suggest that stress is a health crisis associated with several diseases such as cardiovascular diseases, anxiety, and depression. Stress can be defined as a nonspecific bodily response to a combination of external demands and internal concerns. Such a response can be distinguished in the concept of eustress (positive stress) and distress (negative stress). Eustress occurs when a perceived ability to cope outweighs his or her perceived external demands. It can help an individual to increase his or her creativity and productivity and motivate him or her to perform a specific task. However, distress occurs when there is an imbalance between an ability to cope and the perceived external demands. This sort of stress is dangerous and can cause negative consequences. In daily life, the term “stress” is used to describe negative stress rather than positive stress. For the example implementations described herein, the term “stress” is with respect to negative stress.

Measuring the stress level of a human subject can be done in various ways. Stress levels indicates that a user could be very nervous, or very anxious. The human body generally employs the fight-or-flight response to make decisions during a stressful situation instantaneously. The nervous system is responsible for executing this strategy in three consecutive steps: sensing, perceiving, and responding to stress. The most used (traditional) way is to perform the psychological evaluation, usually conducted by handling out clinical stress questionnaires such as the Stress Self Rating Scale. However, this approach is not realistic to conduct under driving or working scenarios. It is well-known that the autonomic nervous system regulates physiological stress responses through the sympathetic nervous system (SNS) and the parasympathetic nervous system (PNS). These physiological stress responses can be measured through different bio-signals (biochemical or physiological). Biochemical signals could be the level of adrenaline and cortisol in various body fluids such as blood, saliva, or urine, as stressful situations increase the level of these hormones in the body. On the other hand, physiological signals can be expressed as a sudden increase of heart activity and/or respiration activity, since the body needs oxygen to be delivered faster to the organs to react quickly. In addition to the bio-signals, external signals such as facial expression can also be used to estimate stress levels since negative emotions such as anger and disgust could be introduced during this process.

Therefore, different systems and frameworks have been developed to detect a stress level based on the above techniques. Driving scenarios gained most of the attention due to the huge loss of traffic accidents caused by stressed drivers every year as well as the feasibility to set up such a system in a vehicular environment. Different research groups have adopted multiple approaches while relying on a single sensor such as an optical camera, photoplethysmogram (PPG), and electrocardiogram (ECG). The consensus has recently been reached that fusing data across multiple sensors is much more effective and accurate than using a single sensor. Further, frameworks utilizing the sensor fusion approach has been developed and demonstrated with credible performance.

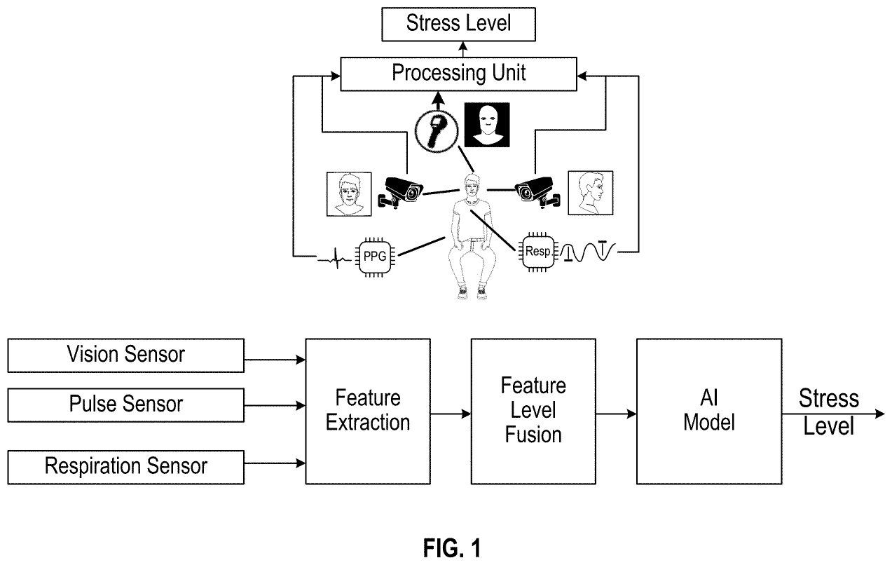

FIG. 1 illustrates a sensor fusion stress monitoring system as implemented in the related art. Different kinds of sensors, including a mono-camera, thermal camera, pulse sensor, and respiration sensor, are used to collect physiological, physical, and facial information. Such information is further processed to sophisticated features. For example, as introduced in the prior art search section, heart rate variability can be extracted from raw heart rate signal and facial expressions extracted from camera-collected images. Those features are further fed into an AI model for stress classification as follows.

The stress classification method can be described with the process of building a function to find the internal relationship between stress level and the input features:

Stress=f(feature-1,feature-2, . . . ,feature-n) (1)

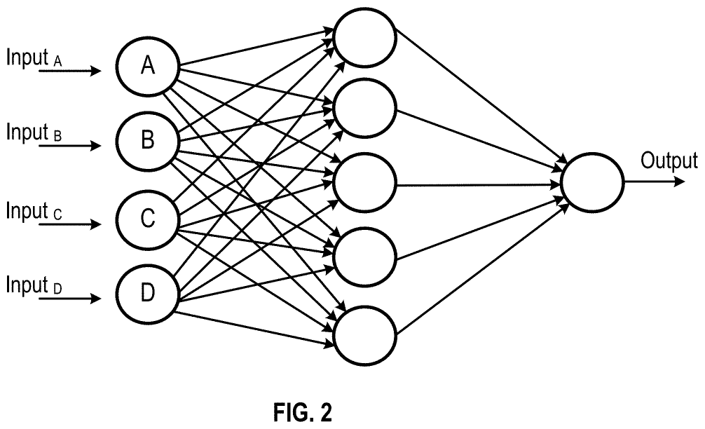

Related art implementations facilitate such methods by a statistically fitted curve to establish a semi-empirical equation. However, with the advancing of AI, this approach has been replaced by training a neural network. FIG. 2 illustrates an example of a neural network. The methods to train a neural network are known extensively in the related art, and the techniques utilized herein can be any of those as known in the related art. A neural network utilizes neurons and the connection between them to represent the non-linear relationship between inputs and output(s), as illustrated, for example in FIG. 2. Specifically, for a sensor fusion stress monitoring system, the AI model is split into different channels (subnetworks) to process features input from various sensors.

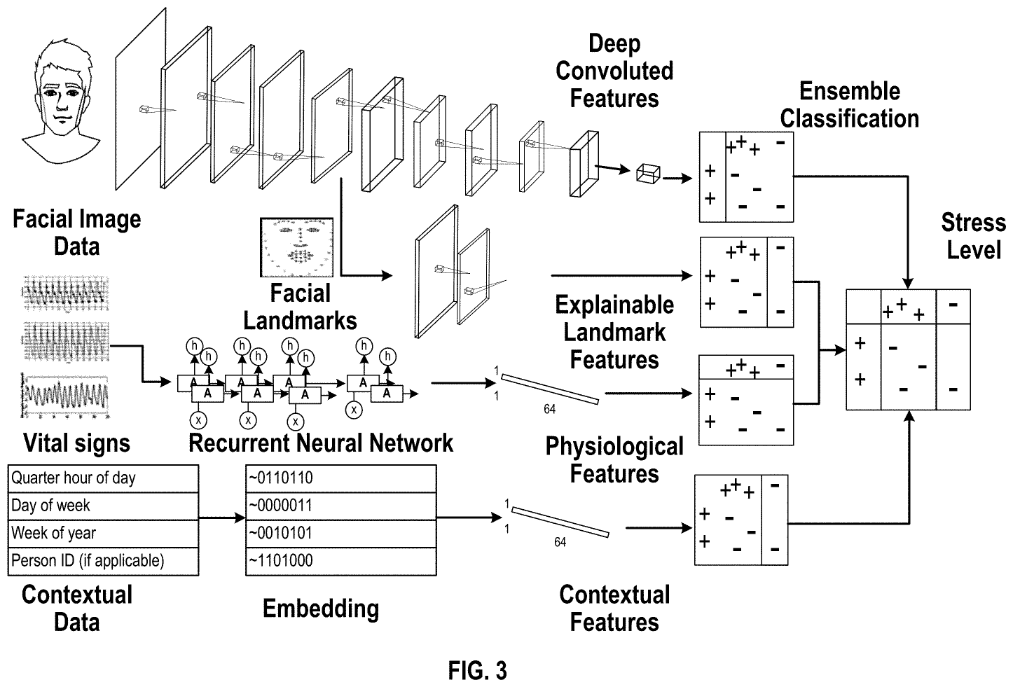

FIG. 3 illustrates an example AI model for a sensor fusion stress monitoring system, in accordance with an example implementation. In FIG. 3, a Convolutional Neural Network (CNN) channel is used to process facial image features. Facial detection is first conducted on the collected frame from the camera where the facial image was extracted as the region-of-interest. Followed by a series of convolution and pooling operations, the information from the original 2D Red Green Blue (RGB) image of facial area was condensed into two 1D vectors. The first vector represents the deep convoluted features directly from the image while the second vector was acquired by analyzing the relative positions of facial landmarks (e.g., eyes, nose, chin). Similarly, vital sign information such as pulse and respirational rate is collected from sensors via ECG, PPG, or remote-PPG techniques. Temporal signal processing techniques are first applied to the raw signal to remove noise. Such techniques include but are not limited to baseline removal, low/high pass filter, and peak detection.

The filtered signals are then fed into a Recurrent Neural Network (RNN) to reveal the sequential relation of each data point with regard to time, and the output is still a vector. Finally, additional contextual data can also be utilized in the sensor fusion deep learning model. A mechanism called embedding is used to convert the natural language words into 1D sequence that a computer can recognize. For example, hours of the day can be encoded as 0 to 23 in decimal and later transformed to a binary number between 0b00000000 to 0b00010111. Therefore, all raw signals from different sensors are converted to vectors which is ready for the fusion process.

The fusion process is conducted via ensemble classification. How to conduct the ensemble approach has been known in the related art with various implementations, and any such implementation known in the related art can be applied in accordance with the desired implementation. In general, three approaches are used predominantly. As indicated in FIG. 3, a majority weighting approach is used to infer the stress level from the weighted sum of the output features, so that:

Stress=w₁×output₁+w₂×output₂+w₃×output₃ (2)

In related art systems, the weights w are either equal or determined empirically.

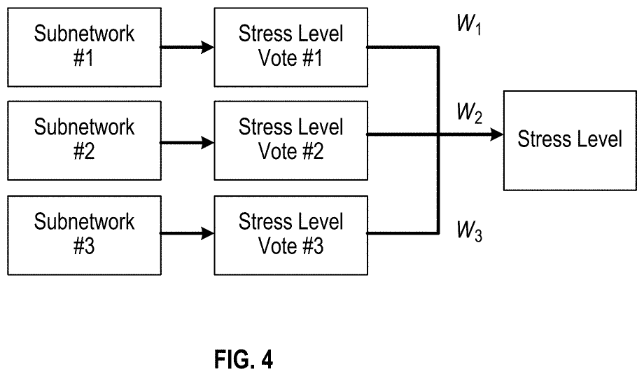

FIG. 4 illustrates a majority weight voting process as an example of ensemble, in accordance with an example implementation. As illustrated in FIG. 4, the various stress level networks are aggregated to a vote to determine stress level. There are also other ways for the ensemble, such as bagging or boosting, which also involves a certain way to allocate weight to each subnetwork.

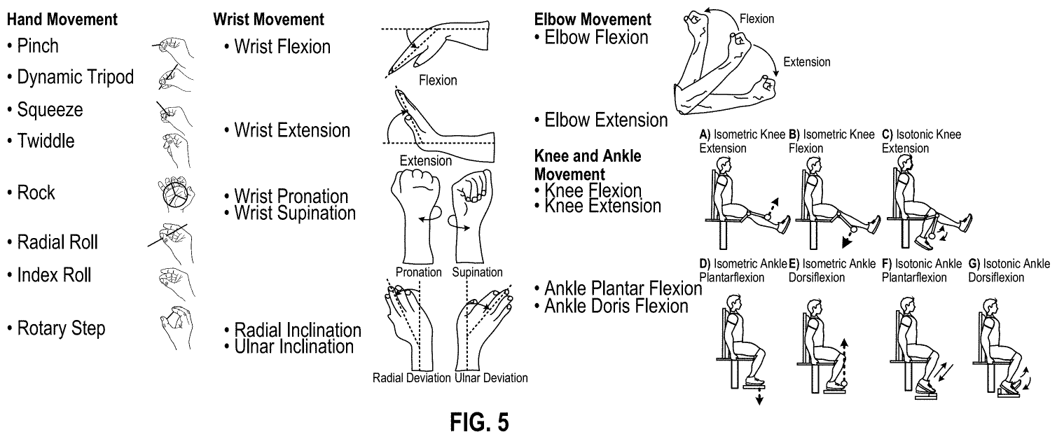

FIG. 5 illustrates examples of human body postures. Recognizing the current human body posture and activity is one of the ways that could provide scene-understanding. Human postures refer to the arrangement of the body and its limbs, which are a base of actions and is often taken as the critical part in various action recognition algorithms. Postures are different from actions, with the former being static and the latter dynamic.

FIG. 5 illustrates several examples of human body postures, where they are represented as angles between different body joints. For example, wrist flexion can be recognized as −30° bending of the wrist joint, while wrist extension is 45°. Apart from the degree, a force can also be exerted on body parts to maintain a posture. Hence, body posture positions are defined as:

Posture=[degree_{hand},degree_{wrist},degree_{elbow}, . . . ,degreeₖₙₑₑ] (3)

There are several main methods for posture recognition. One is to use wearable sensors, such as accelerometer and pressure sensor. The other one is based on cameras and perform image classification to acquire such information. Additionally, posture recognition can be conducted through the seating position via an adjustable seat. In such an example implementation, the adjustable seat determines the human posture, and the seat can be adjusted according to the posture suggestions.

From human postures, activities can be further derived. Consider the running activity. It is composed of repeated postures such as extension and flexion of knee and elbow. Thus, it is possible to establish the connection between activity and postures:

Activity=Posture₀,Posture₁,Posture₂, . . . Postureₜ (4)

where t represents time.

In example implementations described herein, the body posture is utilized as the AI model dynamic feedback. Related art stress monitoring systems are designed to be generic and not context-aware. In other words, related art implementations cannot recognize the scenario and activity of the monitored human subject. Such deficiencies undermine the accuracy. Moreover, a feedback loop is also missing where stress-relieving measures can be recommended if high-level stress is detected. In some related art implementations, a breath metronome is used to help the user regain a regular breathing pattern. However, this kind of approach is far from ideal.

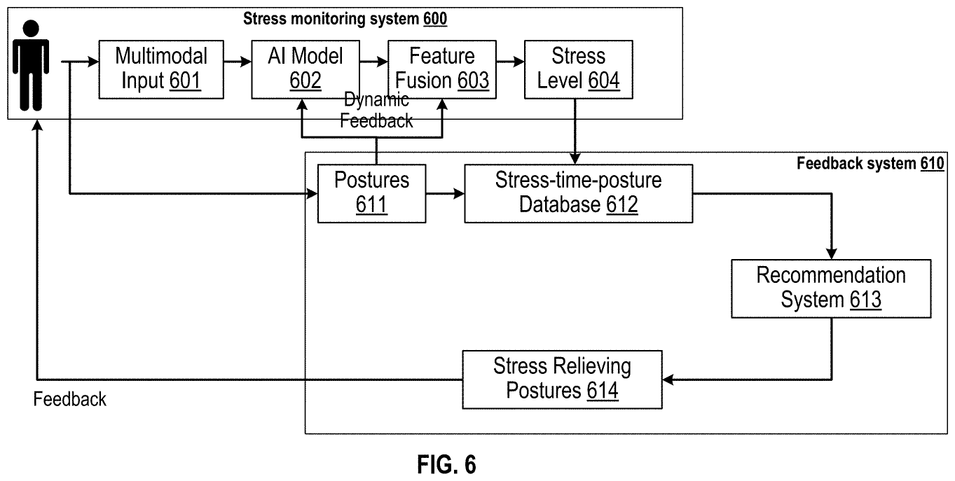

FIG. 6 illustrates an example implementation of a system architecture. The example system involves a stress monitoring system 600, and a feedback system 610. The stress monitoring system 600 processes multimodal input 601 (e.g., camera sensors, wearables, seat sensors, etc.) through an AI model 602, which through the use of feature fusion 603 determines the stress level 604.

To address the related art limitations, human body posture information 611 is added for a feedback system 610. The posture information first serves as dynamic feedback to the AI model 602, specifically for adjusting the weight value of the ensemble classification process. Referring to equation (2) and FIG. 4, the weights are determined empirically or equally to fit a general scenario. With the introduction of body postures, those weights can be determined by another AI model trained by the posture data. Once the stress level 604 is determined through dynamic feedback from the body posture information 611, the body posture information 611 and the stress level 604 are provided through a stress-time-posture database 612, the output of which can be provided to a recommendation system 613 to determine potential stress reliving postures 614.

As illustrated in FIG. 6, the posture of the user is utilized as additional data input to increase accuracy. The machine learning model weights can be adjusted based on the additional data input of the posture, in which the machine learning model adopts the posture as an additional weighted data channel to adjust its accuracy.

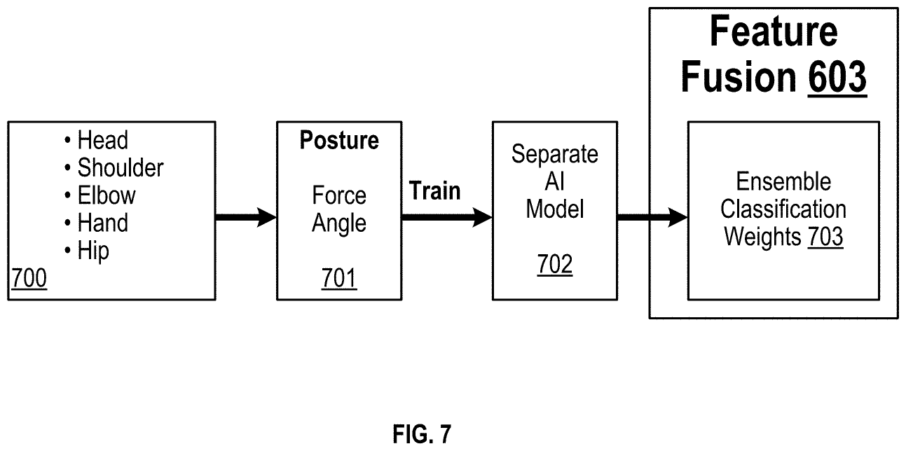

FIG. 7 illustrates an example of utilizing posture as dynamic feedback to the stress classification AI model feature fusion process, in accordance with an example implementation. Specifically, FIG. 7 shows one example of using the force and angle values 701 as measured from each body joint 700, wherein a separate machine learning model 702 is trained to determine the weights 703 under different circumstances. In other words, the weights will be different while the monitored person is sitting and standing.

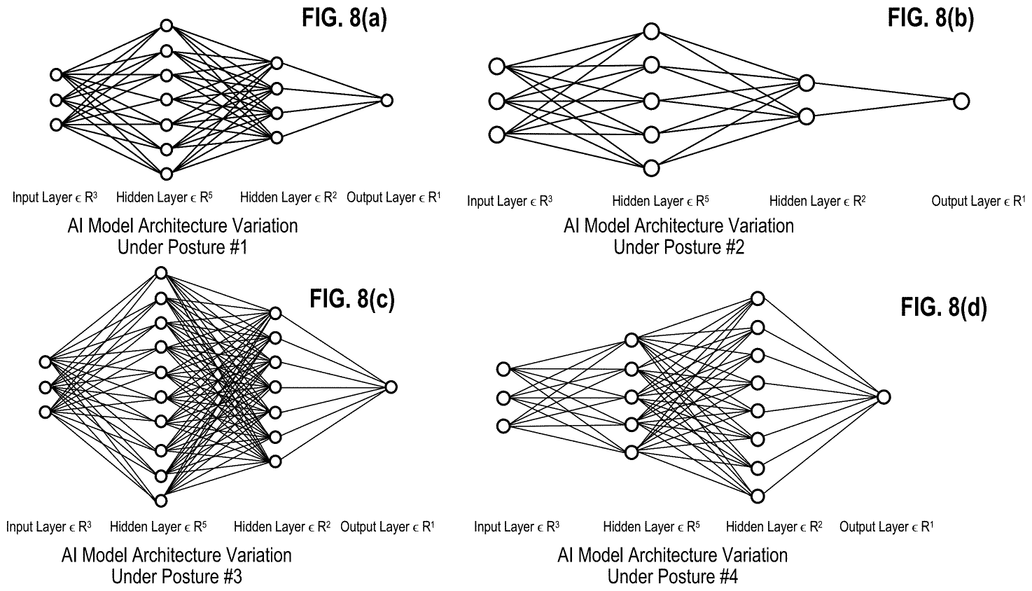

FIGS. 8(a) to 8(d) illustrate example changes to the AI model architecture under dynamic feedback from postures, in accordance with an example implementation.

Another area that dynamic feedback mechanisms can take into action is the neural network architecture variation. Instead of using a fixed neural network architecture for every scenario, a dynamic feedback mechanism can adjust the neurons on each layer to make the architecture more volatile, as shown by four examples demonstrated in FIGS. 8(a) to 8(d). Other feedback mechanisms can also be conducted, including but not limited to dynamically increasing/decreasing layers in the neural network and quantization of the input. As illustrated in FIGS. 8(a) to 8(d), network prunings can be conducted, which as described herein can be pruned according to the force and angles of each body part to determine which way the architecture should be changed. The architecture can also be adjusted based on the different applications of the user (e.g., office versus driving).

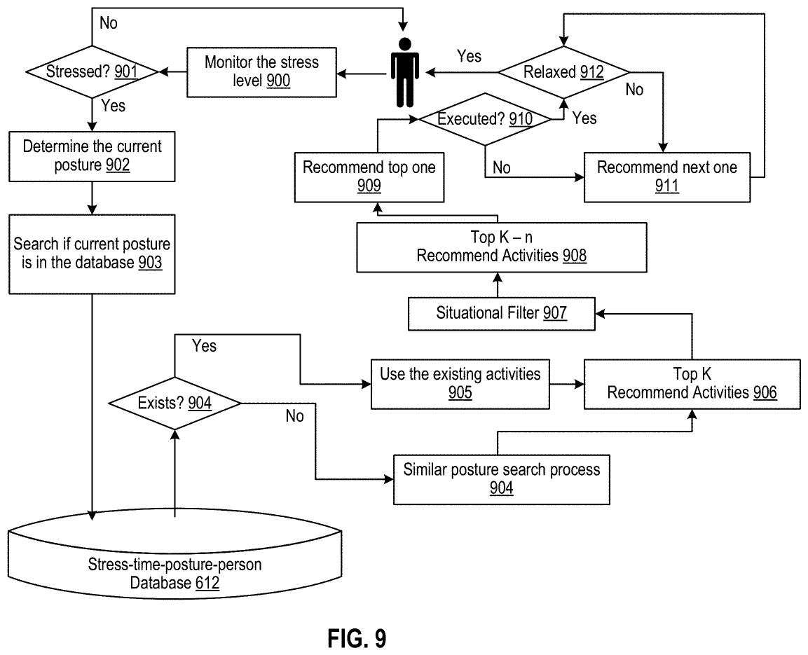

FIG. 9 illustrates an example flowchart to recommend a relaxing posture adjustment based on the posture-stress-time database, in accordance with an example implementation. Once the body posture information is available, a stress-posture-time curve can be obtained for each person, which gives the information on how stress changes over time with varied postures. Therefore, stress relaxing activities can be recommended based on a stress-posture-time-person database; such a process is depicted in FIG. 9.

As shown in FIG. 9, the activation of stress management systems depends on the monitored stress level 900. At 901, if the stress level is indicative of not being stressed (no), then monitoring is continued. Otherwise (yes), the system actively searches for relaxing postures/activities. At 902, the system determines the current posture, so that the searching process can find that given the current posture, whether a relaxing activity has already been recorded in the database at 903. For instance, if it is known that this person can get maximum stress reliving by doing stretching while sitting for a long time, stretching would be directly recommended. There could also be a case that the current posture is not in the database, then a similar posture search process would be conducted, which will be described later herein. If the posture does not exist (No) then the flow proceeds execute a similar posture search process at 904 to determine similar postures, otherwise (Yes), the flow proceeds to use the postures that were found in the database at 905.

Considering one recommendation is commonly not enough, a top K selection is generated in most scenarios at 906. The next step is a situational filter at 907. This filter aims to eliminate activities that are not suitable for the current context. For example. if a person is driving with a sitting posture, stretching would not be a potential choice for stress relaxation. Thus, the recommended activities are narrowed from K to K−n. Once filtered, the top recommendation is provided to the user at 909.

At 910, a determination is made as to whether the posture adjustment activity was executed. Another consideration is that the human subject under monitoring may reject the relaxing activity by directly interacting with the system or simply not following the suggested choice. In this scenario, the system should be able to determine whether the recommended activity has been executed and redirect to the next choice 911 if the result is negative (No). Consequently, it could also happen that the recommended activity is not effective, which resulted in a non-decreasing stress level. Thus, if it is determined that the posture adjustment activity did not reduce stress at 912 (No), the system switches to the next best choice under this circumstance at 911.

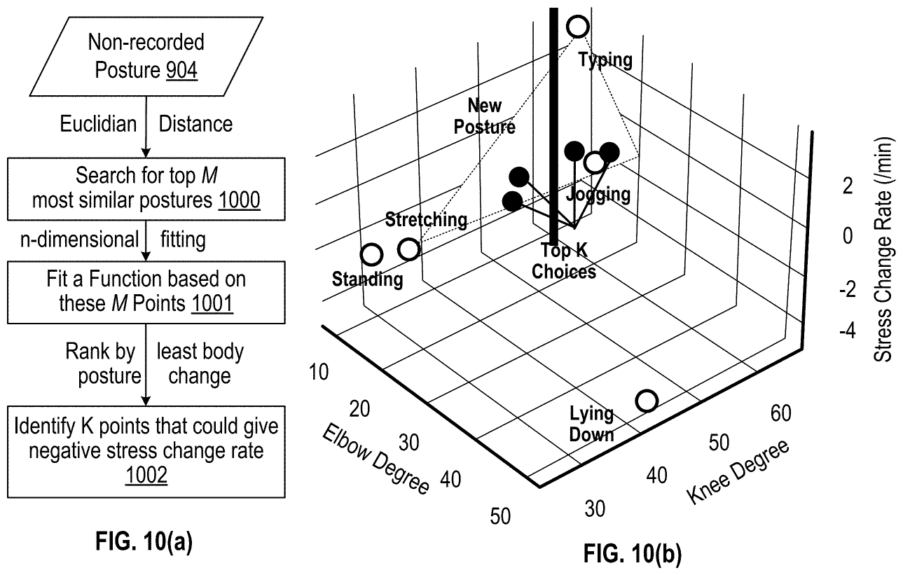

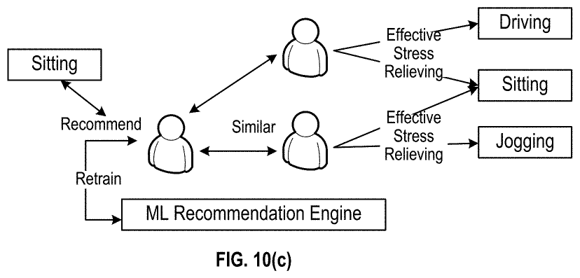

FIGS. 10(a) to 10(c) illustrate a flowchart and an example for a similar posture search process, in accordance with an example implementation. With respect to the similar posture search 904, the process is activated if the current human subject posture is not recorded in the database, which could happen at the initial stage of monitoring when the database contains minimal data.

As indicated in FIG. 10(a), the search process starts at 1000 with the first identified M existing points closest to the current posture by evaluating the Euclidian distance. Once those M points have been identified, an n-dimensional surface can be fitted based on them at 1001. And from this fitted surface, a maximum of K points with a minimum distance of d between each other can be further extracted at 1002, giving a negative stress change rate and close to the current posture.

A visual example is given in FIG. 10(b) with an example involving two dimensions (elbow and knee degree), although there can be over ten dimensions depending on the desired implementation. The existing database has already recorded the stress change rate for activities such as typing, jogging, stretching, standing, and lying down. And when a new posture, represented by the blue pillar, was used to search similar postures, the closest three points determined are typing, starching, and jogging. Thus, a multi-dimensional surface is fitted based on these three existing points. And finally, the top choices are given on the fitting surface that could negatively impact the change rate while the ensuing small posture changed.

FIG. 10(c) illustrates an example flow diagram for addressing when new users are detected in the system, in accordance with an example implementation. Specifically, FIG. 10(c) illustrates an example flow for similar person attribute searching, which is directed to situations in which a new user having insufficient posture information in the database is detected.

In the example of FIGS. 10(a) and 10(b), posture information from the database is utilized for a user having posture information already stored in the database. In such implementations, even if the particular posture of the user does not exist in the database, a process can be executed to determine similar postures for the user by searching the closest data points and conducting function fitting. However, if a completely new user utilizes the current system, then example implementations can utilize a user-based collaborative filtering approach by finding users with similar profiles and conducting majority voting.

In the example of FIG. 10(c), for each new user, the flow finds other users that are similar to the new user from the attribute level (e.g. weight, height, gender, occupation). The similarity can be measured in accordance to known algorithm techniques such as cosine similarity metrics or otherwise in accordance with an example implementation. Once a number of users that are similar to this person are found, the common stress-reducing posture/activities can thereby be identified and recommended to the new user.

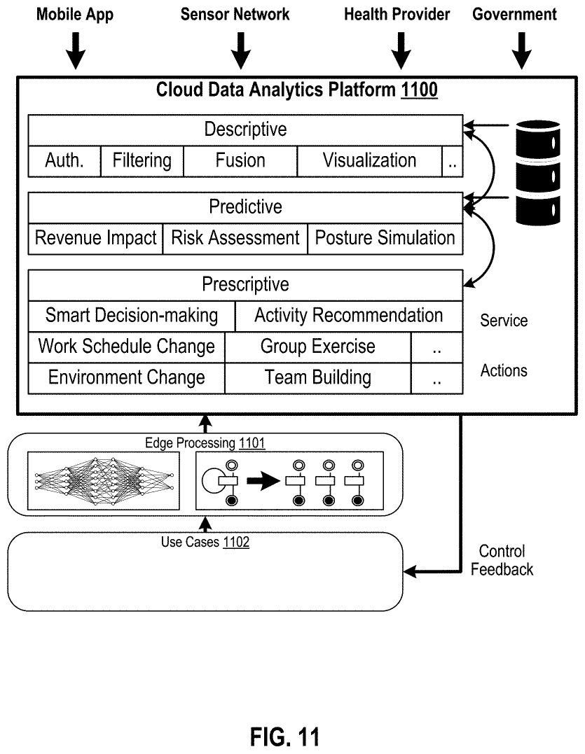

FIG. 11 illustrates the system architecture of the cloud-enabled human stress monitoring and management system, in accordance with an example implementation. Cloud computing has been a leading trend in digital innovation, as the business of all sizes has been moving the computational-heavy tasks to the cloud side to reduce loads on the edge side. For the example implementations described herein, recommending relaxing activities is a data-intensive application and requires all individual data collections to make the decision. Therefore, it is natural that the stress management application should be moved to the cloud side. As for the monitoring application, processing data in real-time becomes the primary requirement, and latency between edge-cloud-edge data transferring could limit the data processing capability. Hence, example implementations can be deployed on the edge side for edge processing 1101.

FIG. 11 illustrates the architecture of the proposed system that is fitted for multiple applications including autonomous shuttle, construction machinery, and office working. In the proposed architecture, the AI model is deployed on the edge side to monitor the stress level of the persons and subject to dynamic feedback from the posture positions. In order to achieve that, sensors are deployed on places such as the ceiling, walls, safety belt, and seat, and they are used to collect the necessary information from the human subject. The sensory information is preprocessed and fed into the end-to-end AI model to infer the stress level as described above. In the meantime, the body posture is collected from the existing sensors as well, and both stress level information and body posture are sent to the cloud database for further analysis.

All stress levels and postures are aggregated in a cloud database, referred to as the cloud data analytics platform depicted in FIG. 11. Once a high-stress level has been transferred to the cloud, the stress management system in the prescriptive analytics would be activated to find out if there were any stress-relieving measures, such a process has been described in the previous subsection and outlined in FIG. 9 and FIGS. 10(a) to 10(c). Some of the changes, including recommending individual postures such as sitting down or jogging, change the work schedule to give more resting time, or change the environment to play music.

Another aspect of a cloud-based data analytics platform 1100 is visualization. Managers or administrators can check how the stress level is spread out in different departments and geographical areas. The cloud platform 1100 can also assess how the current collected stress level changes would impact revenue and risk. The cloud platform 1100 is also capable of conducting prediction and simulation tasks. For example, it can predict based on the current stress level change, how much would the health risk be and how corporation revenue would be impacted by the collective stress level. It could also simulate how the change of posture could virtually affect the current stress level based on the existing data.

Finally, the platform is also capable of communication with other entities across various use cases 1102 due to its connectivity nature. End users could interact with a mobile app to track their personal stress level and posture recommendations. Administrators could visualize the simulation and prediction results via the same terminal. The collected information can also be shared with heath service provider and government agencies to ensure the safety of the operations.

In the following, there are various example scenarios to illustrate how the application can achieve context-awareness. One example use case 1102 is monitoring a driver of a motor vehicle.

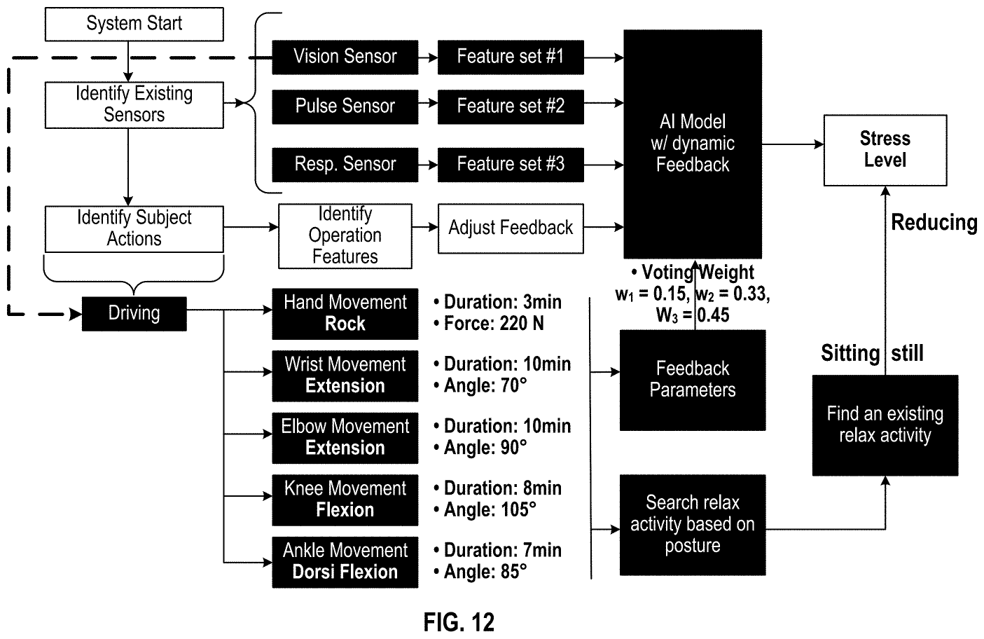

FIG. 12 illustrates the application aware stress monitoring and management as applied to driving, in accordance with an example implementation.

In this scenario, a camera is placed in front of the vehicle to capture the driver facial images, and sensors are placed in the seat and steering wheel to collect pulse and respirational rate information. Additional sensors are also placed inside the vehicle to understand the current posture, including the angle and forces exerted on each joint. From the measured posture parameters, dynamic feedback is first applied to the AI model, where voting weight is determined by a separate model and used to adjust the weight of each feature set. Since the current posture (uncomfortable sitting) is stressful, the stress management system starts to search in the database where relaxing postures exist. Based on the previous data collected, the system suggests that the driver sits up straight to relieve the stress. Once the driver can execute the recommendation and the stress level returned to normal, the recommendation process is stopped. Otherwise, another resting posture can be recommended, such as positioning knees at the same height or slightly lower than the hips.

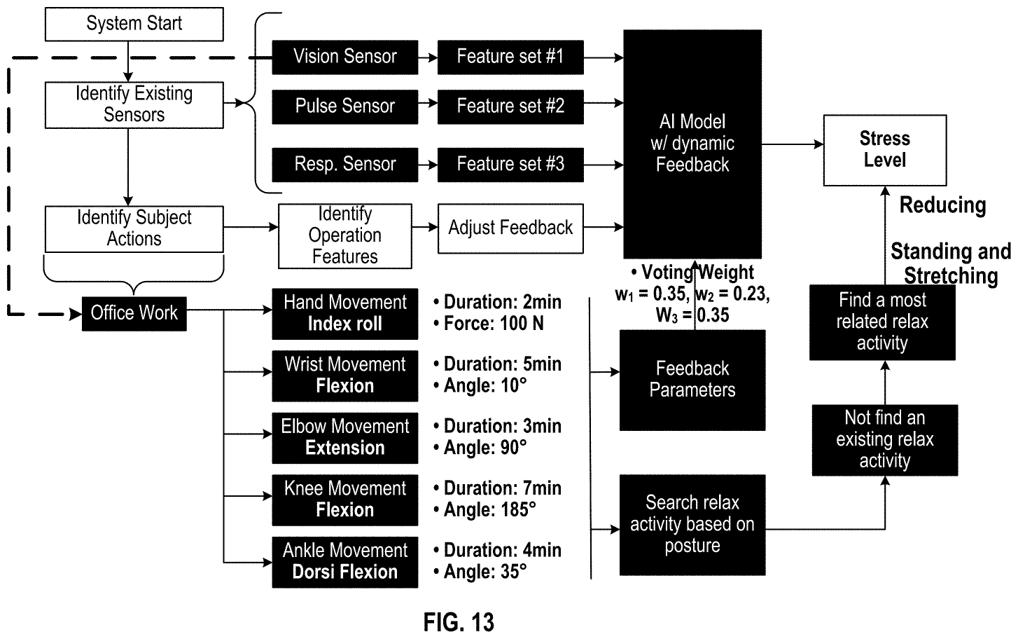

FIG. 13 illustrates the application aware stress monitoring and management as applied to desk working, in accordance with an example implementation. In this second example, an office worker is detected as stressed through laptop embedded cameras and smart wearable devices. In addition, the posture is determined by a separate set of sensors deployed on the office ceiling and keyboard. A similar dynamic feedback is conducted to adjust the voting weight of each AI model channel, where the results are different from those of the driving scenario. Due to the current stressful state, the stress management system starts to search for a posture to relax but could not find an existing database solution. Therefore, a similar posture searching process is conducted following the procedure described in FIGS. 10(a) to 10(c). After the posture of standing and stretching is recommended to the office worker, the system will continue monitoring the stress level if it did not fall as expected.

Thus, through the example implementations described herein, postures are utilized as additional data input to increase accuracy of AI model systems for determining stress levels. The posture can be a dynamic feedback mechanism to alter the neural network architecture, including but not limited to change neurons in each layer, change the number of layers, conduct network quantization, and so on.

Further, the posture can be used as a dynamic feedback mechanism to the ensemble classification process, including but not limited to changing the voting weight of each channel, changing how the dataset is sampled during the bagging process, and changing how data points are weighted during the boosting process.

Example implementations can further store posture-stress-time curves in the database to recommend personalized stress-relieving postures, and find an existing reliving activity if the current posture is recorded in the database. Example implementations can find a similar relieving activity if the current posture is not recorded in the database. Example implementations can also use a cloud-edge architecture for the proposed system where the stress management application is running on the cloud, and the stress monitoring application is deployed on the edge and receiving feedback suggestions.

The example implementations described herein can be applied as generic usage for different applications. It can be employed for a future smart city in sectors such as manufacturing, mobility, healthcare, and office. For example. it can be deployed in a vehicle where passengers can be monitored by an internal camera, seat, and safety belt sensors. It can also be used in an office working scenario where smart wearable devices can collect information. Stress is still one factor that impacts work performance and mental health. Both end customers and corporations need an accurate stress management solution.

Further, the example implementations described herein can extend the machine learning model to detect a wellness state of the user (e.g., drowsiness, irritation or other emotional states, etc.), and the recommending the one or more postures to the user is conducted based on the detected wellness state. The machine learning model can be configured to be trained to detect such wellness states through any method as known in the art, wherein the posture recommendation can then be adjusted to address the wellness state as needed. In an example, drowsiness can be addressed by a recommendation of a posture that reduces drowsiness while further, during a driving situation, providing a message for the user to pull over.

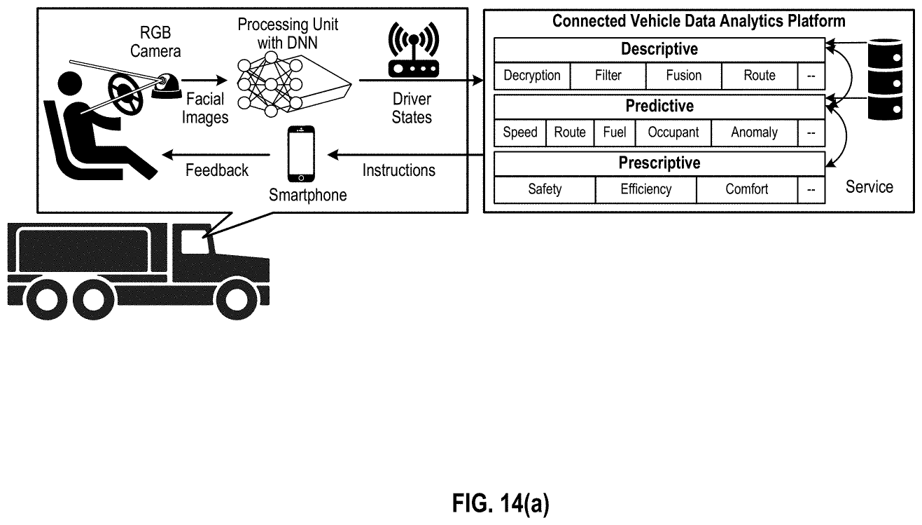

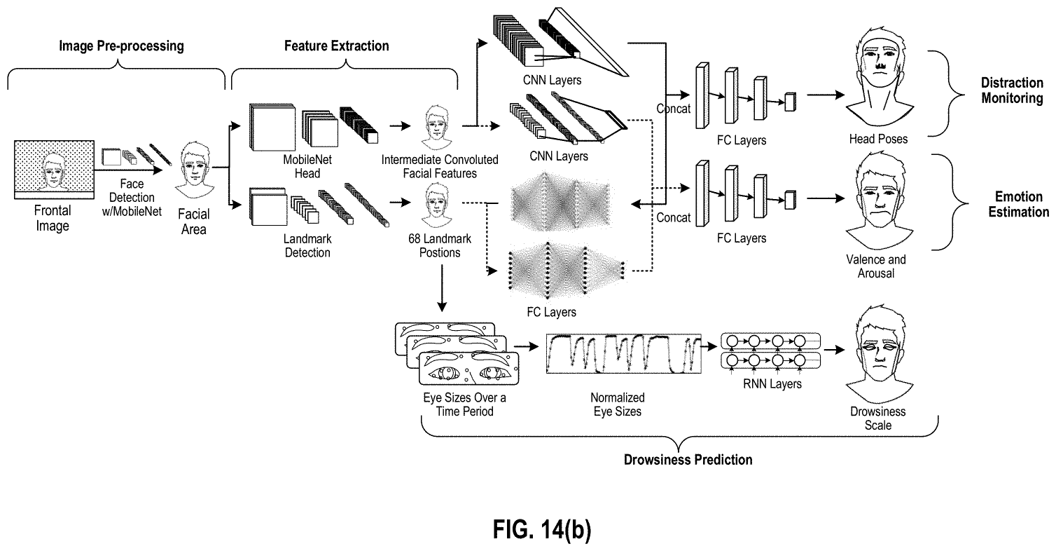

FIGS. 14(a) and 14(b) illustrate an example processing application to determine driver states, in accordance with an example implementation. As illustrated in FIG. 14(a), example implementations for the edge system can involve an RGB camera, a processing unit configured with a deep neural network (DNN), and a network that facilitates a connection to the cloud platform. The cloud platform can transmit instructions to the user to a user device such as a smartphone. The processing unit can be any type of hardware processor as known in the art, such as, but not limited to, electronic control units (ECUs), tensor processors, graphical processing units (GPUs) and so on to configure the vehicle to be an edge system.

FIG. 14(b) illustrates an example of the architecture facilitated by the edge processing unit, in accordance with an example implementation. The RGB camera from FIG. 14(a) captures a frontal image, from which the processing unit executes image pre-processing to conduct facial extraction. Feature extraction can utilize techniques such as, but not limited to, MobileNet Head and landmark detection to generate intermediate convoluted facial features and landmark positions. The intermediate convoluted facial features are processed by CNN layers and FC layers jointly with processing the landmark positions of the machine learning model to facilitate distraction monitoring as well as emotion estimation. Further, a drowsiness prediction model can be constructed by RNNs trained against normalized eye sizes over a time period to determine a level of drowsiness by the user.

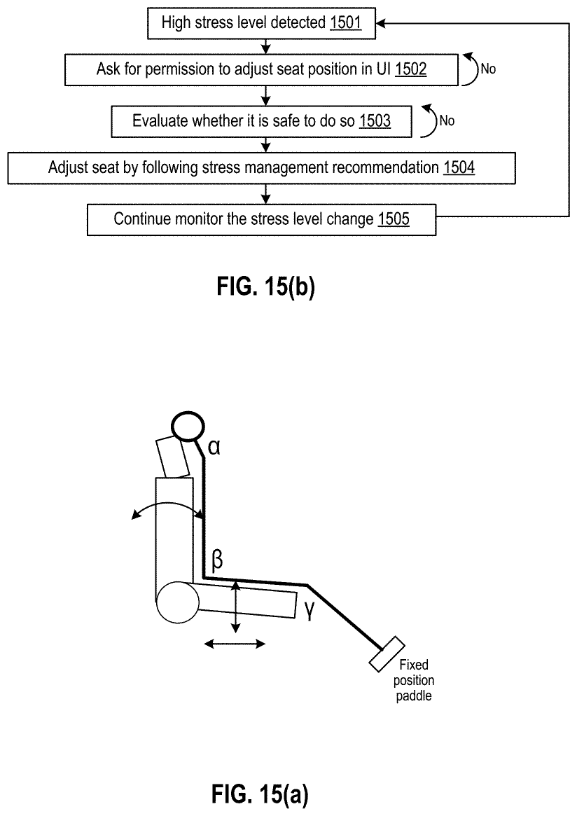

FIG. 15(a) illustrates an example of an automatically adjustable seat, in accordance with an example implementation. In example implementations, the system can be configured to automatically help change the posture by adjusting seat positions and re-evaluate the stress level change, while taking into consideration the user safety and permission from the user. Further, the parameters of the seat position can also be utilized for determining or measuring the posture of the user at any given point in time.

FIG. 15(b) illustrates an example flow diagram of the automatically adjustable seat, in accordance with an example implementation. The flow starts when a high stress level (e.g., above a threshold) is detected at 1501, a user interface can interact with the user to receive permission from the user to adjust the seat position at 1502. If so, then the flow proceeds to 1503 to determine if it is safe to adjust the seat position. The safety of the adjustment of the seat position can be based on any function in accordance with the desired implementation, considering factors such as, but not limited to, vehicle speed, posture of the user, location of the vehicle, and so on. Once it is evaluated as being safe for adjustment, the flow can then proceed to 1504 to adjust the seat based on the stress management recommendation as provided by the models described herein. Once complete, the system can revert back to continual monitoring of the stress level change of the user at 1505.

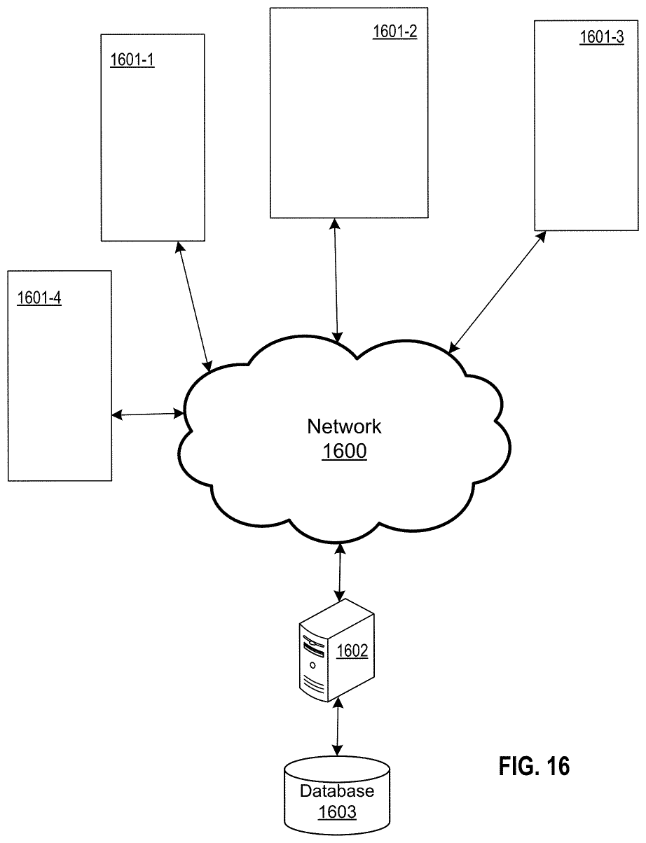

FIG. 16 illustrates a system involving a plurality of edge systems with connected sensors and a management apparatus, in accordance with an example implementation. One or more edge systems 1601-1, 1601-2, 1601-3, and 1601-4 are equipped with sensors and are communicatively coupled to a network 1600 which is connected to a management apparatus 1602, which facilitates functionality for an Internet of Things (IoT) gateway or other management system. The management apparatus 1602 manages a database 1603, which contains historical data collected from the sensor systems 1601-1, 1601-2, 1601-3, and 1601-4, which can include labeled data and unlabeled data as received from the systems 1601-1, 1601-2, 1601-3, and 1601-4. In alternate example implementations, the data from the edge systems 1601-1, 1601-2, 1601-3, 1601-4 can be stored to a central repository or central database such as proprietary databases that intake data such as enterprise resource planning systems, and the management apparatus 1602 can access or retrieve the data from the central repository or central database. Database 1603 can also manage the posture-stress-time database as illustrated in FIG. 9 to record how stress level has changed among various user postures. Such systems can include sensors connected with transmission devices to facilitate communication from monitoring users while driving, while on a desk, and so on in accordance with the desired implementation. Such sensors can involve vision sensors such as a camera capturing facial signals, a pulse sensor capturing heart beat rate, respiratory sensors capturing the breathing rate, and so on.

Each of the edge systems 1601-1, 1601-2, 1601-3, 1601-4 can be configured to provide the posture recommendation via a visualization (e.g., on a display), by audio recommendations, and so on depending on the nature of the edge system. For example, if the edge system is a vehicle, then the posture recommendation can be made via an audio call out (e.g., voice indicating please move your arm lower), or by an adjustment to the seat as illustrated in FIGS. 15(a) and 15(b). In an office scenario, a display can be used to provide a visual recommendation. The output of such recommendations can be adjusted in accordance with the desired implementation.

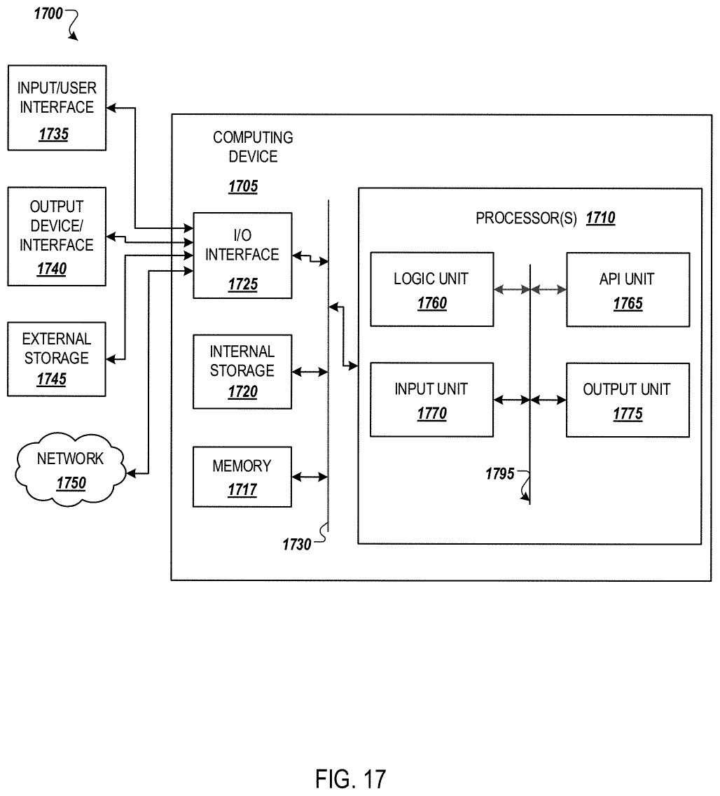

FIG. 17 illustrates an example computing environment with an example computer device suitable for use in some example implementations, such as a management apparatus 1602 as illustrated in FIG. 16 or as a device to facilitate an edge system as illustrated in FIG. 14(a).

Computer device 1705 in computing environment 1700 can include one or more processing units, cores, or processors 1710, memory 1715 (e.g., RAM, ROM, and/or the like), internal storage 1720 (e.g., magnetic, optical, solid state storage, and/or organic), and/or I/O interface 1725, any of which can be coupled on a communication mechanism or bus 1730 for communicating information or embedded in the computer device 1705. I/O interface 1725 is also configured to receive images from cameras or provide images to projectors or displays, depending on the desired implementation.

Computer device 1705 can be communicatively coupled to input/user interface 1735 and output device/interface 1740. Either one or both of input/user interface 1735 and output device/interface 1740 can be a wired or wireless interface and can be detachable. Input/user interface 1735 may include any device, component, sensor, or interface, physical or virtual, that can be used to provide input (e.g., buttons, touch-screen interface, keyboard, a pointing/cursor control, microphone, camera, braille, motion sensor, optical reader, and/or the like). Output device/interface 1740 may include a display, television, monitor, printer, speaker, braille, or the like. In some example implementations, input/user interface 1735 and output device/interface 1740 can be embedded with or physically coupled to the computer device 1705. In other example implementations, other computer devices may function as or provide the functions of input/user interface 1735 and output device/interface 1740 for a computer device 1705.

Examples of computer device 1705 may include, but are not limited to, highly mobile devices (e.g., smartphones, devices in vehicles and other machines, devices carried by humans and animals, and the like), mobile devices (e.g., tablets, notebooks, laptops, personal computers, portable televisions, radios, and the like), and devices not designed for mobility (e.g., desktop computers, other computers, information kiosks, televisions with one or more processors embedded therein and/or coupled thereto, radios, and the like).

Computer device 1705 can be communicatively coupled (e.g., via I/O interface 1725) to external storage 1745 and network 1750 for communicating with any number of networked components, devices, and systems, including one or more computer devices of the same or different configuration. Computer device 1705 or any connected computer device can be functioning as, providing services of, or referred to as a server, client, thin server, general machine, special-purpose machine, or another label.

I/O interface 1725 can include, but is not limited to, wired and/or wireless interfaces using any communication or I/O protocols or standards (e.g., Ethernet, 802.11x, Universal System Bus, WiMax, modem, a cellular network protocol, and the like) for communicating information to and/or from at least all the connected components, devices, and network in computing environment 1700. Network 1750 can be any network or combination of networks (e.g., the Internet, local area network, wide area network, a telephonic network, a cellular network, satellite network, and the like).

Computer device 1705 can use and/or communicate using computer-usable or computer-readable media, including transitory media and non-transitory media. Transitory media include transmission media (e.g., metal cables, fiber optics), signals, carrier waves, and the like. Non-transitory media include magnetic media (e.g., disks and tapes), optical media (e.g., CD ROM, digital video disks, Blu-ray disks), solid state media (e.g., RAM, ROM, flash memory, solid-state storage), and other non-volatile storage or memory.

Computer device 1705 can be used to implement techniques, methods, applications, processes, or computer-executable instructions in some example computing environments. Computer-executable instructions can be retrieved from transitory media, and stored on and retrieved from non-transitory media. The executable instructions can originate from one or more of any programming, scripting, and machine languages (e.g., C, C++, C#, Java, Visual Basic, Python, Perl, JavaScript, and others).

Processor(s) 1710 can execute under any operating system (OS) (not shown), in a native or virtual environment. One or more applications can be deployed that include logic unit 1760, application programming interface (API) unit 1765, input unit 1770, output unit 1775, and inter-unit communication mechanism 1795 for the different units to communicate with each other, with the OS, and with other applications (not shown). The described units and elements can be varied in design, function, configuration, or implementation and are not limited to the descriptions provided.

In some example implementations, when information or an execution instruction is received by API unit 1765, it may be communicated to one or more other units (e.g., logic unit 1760, input unit 1770, output unit 1775). In some instances, logic unit 1760 may be configured to control the information flow among the units and direct the services provided by API unit 1765, input unit 1770, output unit 1775, in some example implementations described above. For example, the flow of one or more processes or implementations may be controlled by logic unit 1760 alone or in conjunction with API unit 1765. The input unit 1770 may be configured to obtain input for the calculations described in the example implementations, and the output unit 1775 may be configured to provide output based on the calculations described in example implementations.

Processor(s) 1710 can be configured to determine a posture of a user from data provided from one or more sensors; determine a stress level of the user from a machine learning model configured to determine the stress level of the user based on the data provided from the one or more sensors, for the determined stress level being above a threshold, determine one or more new postures to reduce the stress level from the database of postures; and recommend the one or more postures to the user as illustrated in FIGS. 6 and 9. Depending on the desired implementation, the machine learning model can be configured to be continuously trained from a feedback process incorporating the posture of the user as input as illustrated in FIG. 6.

Depending on the desired implementation, the determining the posture of the user from the data provided from the one or more sensors and the determining the stress level of the user from the machine learning model configured to determine the stress level of the user based on the data provided from one or more sensors are executed on an edge system, and wherein the determining the one or more new postures to reduce the stress level from the database of postures and recommending the one or more postures to the user is executed on a cloud system as illustrated in FIG. 14(a).

Processor(s) 1710 can be configured to determine the stress level of the user from the machine learning model configured to determine the stress level of the user is based on the data provided from the one or more sensors comprises incorporating the posture of the user in a weighted channel of the machine learning model as illustrated in FIG. 4.

Processor(s) 1710 can be configured to recommend the one or more postures to the user by searching a database relating stress, posture, and time to determine a plurality of postures associated with a relaxing activity given the posture of the user; executing a situational filter to filter the plurality of postures to the one or more postures according to a current situation of the user; and recommending the filtered one or more postures as illustrated in FIGS. 10(a) to 10(c).

Processor(s) 1710 can be configured to determine the stress level of the user from the machine learning model configured to determine the stress level of the user based on the data provided from the one or more sensors by extracting features from the data; and assembling the features to determine the stress level based on posture as illustrated in FIG. 9.

Processor(s) 1710 can be configured to detect, from the machine learning model, a wellness state of the user, wherein the recommending the one or more postures to the user is based on the wellness state of the user as illustrated in FIG. 14(b).

Some portions of the detailed description are presented in terms of algorithms and symbolic representations of operations within a computer. These algorithmic descriptions and symbolic representations are the means used by those skilled in the data processing arts to convey the essence of their innovations to others skilled in the art. An algorithm is a series of defined steps leading to a desired end state or result. In example implementations, the steps carried out require physical manipulations of tangible quantities for achieving a tangible result.

Unless specifically stated otherwise, as apparent from the discussion, it is appreciated that throughout the description, discussions utilizing terms such as “processing,” “computing,” “calculating,” “determining,” “displaying,” or the like, can include the actions and processes of a computer system or other information processing device that manipulates and transforms data represented as physical (electronic) quantities within the computer system's registers and memories into other data similarly represented as physical quantities within the computer system's memories or registers or other information storage, transmission or display devices.

Example implementations may also relate to an apparatus for performing the operations herein. This apparatus may be specially constructed for the required purposes, or it may include one or more general-purpose computers selectively activated or reconfigured by one or more computer programs. Such computer programs may be stored in a computer readable medium, such as a computer-readable storage medium or a computer-readable signal medium. A computer-readable storage medium may involve tangible mediums such as, but not limited to optical disks, magnetic disks, read-only memories, random access memories, solid state devices and drives, or any other types of tangible or non-transitory media suitable for storing electronic information. A computer readable signal medium may include mediums such as carrier waves. The algorithms and displays presented herein are not inherently related to any particular computer or other apparatus. Computer programs can involve pure software implementations that involve instructions that perform the operations of the desired implementation.

Various general-purpose systems may be used with programs and modules in accordance with the examples herein, or it may prove convenient to construct a more specialized apparatus to perform desired method steps. In addition, the example implementations are not described with reference to any particular programming language. It will be appreciated that a variety of programming languages may be used to implement the techniques of the example implementations as described herein. The instructions of the programming language(s) may be executed by one or more processing devices, e.g., central processing units (CPUs), processors, or controllers.

As is known in the art, the operations described above can be performed by hardware, software, or some combination of software and hardware. Various aspects of the example implementations may be implemented using circuits and logic devices (hardware), while other aspects may be implemented using instructions stored on a machine-readable medium (software), which if executed by a processor, would cause the processor to perform a method to carry out implementations of the present application. Further, some example implementations of the present application may be performed solely in hardware, whereas other example implementations may be performed solely in software. Moreover, the various functions described can be performed in a single unit, or can be spread across a number of components in any number of ways. When performed by software, the methods may be executed by a processor, such as a general purpose computer, based on instructions stored on a computer-readable medium. If desired, the instructions can be stored on the medium in a compressed and/or encrypted format.

Moreover, other implementations of the present application will be apparent to those skilled in the art from consideration of the specification and practice of the techniques of the present application. Various aspects and/or components of the described example implementations may be used singly or in any combination. It is intended that the specification and example implementations be considered as examples only, with the true scope and spirit of the present application being indicated by the following claims.

What is claimed is:

1. A method, comprising:
determining a posture of a user from data provided from one or more sensors;
determining a stress level of the user from a machine learning model configured to determine the stress level of the user based on the data provided from the one or more sensors;
for the determined stress level being above a threshold:
determining one or more new postures to reduce the stress level from the database of postures; and
recommending the one or more postures to the user.

2. The method of claim 1, wherein the machine learning model is configured to be continuously trained from a feedback process incorporating the posture of the user as input.

3. The method of claim 1, wherein the determining the posture of the user from the data provided from the one or more sensors and the determining the stress level of the user from the machine learning model configured to determine the stress level of the user based on the data provided from one or more sensors are executed on an edge system, and wherein the determining the one or more new postures to reduce the stress level from the database of postures and recommending the one or more postures to the user is executed on a cloud system.

4. The method of claim 1, wherein the determining the stress level of the user from the machine learning model configured to determine the stress level of the user based on the data provided from the one or more sensors comprises incorporating the posture of the user in a weighted channel of the machine learning model.

5. The method of claim 1, wherein the recommending the one or more postures to the user comprises:
searching a database relating stress, posture, and time to determine a plurality of postures associated with a relaxing activity given the posture of the user;
executing a situational filter to filter the plurality of postures to the one or more postures according to a current situation of the user; and
recommending the filtered one or more postures.

6. The method of claim 1, wherein the determining the stress level of the user from the machine learning model configured to determine the stress level of the user based on the data provided from the one or more sensors comprises:
extracting features from the data;
assembling the features to determine the stress level based on posture.

7. The method of claim 1, further comprising detecting, from the machine learning model, a wellness state of the user, wherein the recommending the one or more postures to the user is based on the wellness state of the user.

8. A non-transitory computer readable medium, storing instructions for executing a process, the instructions comprising:
determining a posture of a user from data provided from one or more sensors;
determining a stress level of the user from a machine learning model configured to determine the stress level of the user based on the data provided from the one or more sensors;
for the determined stress level being above a threshold:
determining one or more new postures to reduce the stress level from the database of postures; and
recommending the one or more postures to the user.

9. The non-transitory computer readable medium of claim 8, wherein the machine learning model is configured to be continuously trained from a feedback process incorporating the posture of the user as input.

10. The non-transitory computer readable medium of claim 8, wherein the determining the posture of the user from the data provided from the one or more sensors and the determining the stress level of the user from the machine learning model configured to determine the stress level of the user based on the data provided from one or more sensors are executed on an edge system, and wherein the determining the one or more new postures to reduce the stress level from the database of postures and recommending the one or more postures to the user is executed on a cloud system.

11. The non-transitory computer readable medium of claim 8, wherein the determining the stress level of the user from the machine learning model configured to determine the stress level of the user based on the data provided from the one or more sensors comprises incorporating the posture of the user in a weighted channel of the machine learning model.

12. The non-transitory computer readable medium of claim 8, wherein the recommending the one or more postures to the user comprises:
searching a database relating stress, posture, and time to determine a plurality of postures associated with a relaxing activity given the posture of the user;
executing a situational filter to filter the plurality of postures to the one or more postures according to a current situation of the user; and
recommending the filtered one or more postures.

13. The non-transitory computer readable medium of claim 8, wherein the determining the stress level of the user from the machine learning model configured to determine the stress level of the user based on the data provided from the one or more sensors comprises:
extracting features from the data;
assembling the features to determine the stress level based on posture.

14. The non-transitory computer readable medium of claim 8, further comprising detecting, from the machine learning model, a wellness state of the user, wherein the recommending the one or more postures to the user is based on the wellness state of the user.

15. An apparatus, comprising:
a processor, configured to:
determine a posture of a user from data provided from one or more sensors;
determine a stress level of the user from a machine learning model configured to determine the stress level of the user based on the data provided from the one or more sensors;
for the determined stress level being above a threshold:
determine one or more new postures to reduce the stress level from the database of postures; and
recommend the one or more postures to the user.
